(19) United States
(12) Patent Application Publication
(10) Pub. No.: US 2024/0000738 A1
(43) Pub. Date: Jan. 4, 2024

(54) HYDROXYSTEARIC ACID FOR INDUCING GENERATION OF ANTIMICROBIAL PEPTIDES

(71) Applicant: Conopco, Inc., d/b/a UNILEVER, Englewood Cliffs, NJ (US)

(72) Inventors: Thrisha Cleetus ALEXANDER, Bangalore (IN); Rimpa GHOSH, Kolkata (IN); Amitabha MAJUMDAR, Bangalore (IN); Mruthyunjaya Swamy MATHAPATHI, Bangalore (IN); Sreenivasa THIMMAIAH, Kadur (IN); Morris WASKAR, Bangalore (IN)

(21) Appl. No.: 18/037,372

(22) PCT Filed: Nov. 24, 2021
(86) PCT No.: PCT/EP2021/082733

§ 371 Date: May 17, 2023

(30) Foreign Application Priority Data
Dec. 3, 2020 (IN) 202021052758
Jan. 21, 2021 (EP) 21152775.9

(51) Int. Cl. A61K 31/20; A61K 31/133; A61P 31/04; A61K 38/17; A61K 8/365; A61Q 17/00; A61K 8/64; A61K 8/41

(52) U.S. Cl. CPC A61K 31/20; A61K 31/133; A61P 31/04; A61K 38/1729; A61K 8/365; A61Q 17/005; A61K 8/64; A61K 8/41

(57) ABSTRACT

The invention relates to new use of hydroxystearic acid for inducing secretion of anti-microbial peptides (AMPs) when applied on an external surface of the human body. It also relates to an antimicrobial composition comprising a combination of hydroxystearic acid and antimicrobial peptides or antimicrobial lipids or mixtures thereof. This has application in improving the immunity of skin, scalp and oral cavity against attack by microorganisms.

### FIELD OF THE INVENTION

The invention relates to a new use of hydroxystearic acid for generation of AMPs (anti-microbial peptides) on skin. This has application in improving the immunity of skin, scalp and oral cavity against attack by microorganisms. The invention also relates to use of a combination of hydroxystearic acid and AMPsor AMLs (antimicrobial lipids) or mixtures thereof for antimicrobial benefit and to a composition comprising this combination.

### BACKGROUND OF THE INVENTION

Skin is the primary line of defense that protects the human body from invading pathogens, like virus and bacteria. As a primary defense organ, the skin tissue always remains in constant contact with the environment and therefore it has to face and resolve threats and challenges from invading pathogens. The exposed skin surface is not only challenged by pathogenic foreign bacteria, but it also remains in contact and interacts with the resident commensal bacteria. In spite of all these challenges from such foreign microbes, healthy skin remains infection free and also the numbers of the resident microflora remains generally constant. This equilibrium in the interaction between the skin tissue and the microbes is maintained as the skin has sophisticated defense strategy of which anti-microbial peptides (AMPs) form an important part.

AMPs form an integral part of the skin's own defense system. AMPs are ubiquitous in nature and they typically exhibit a broad spectrum of activity against invading bacteria, fungi, viruses and parasites. AMPs are generally short peptides and in humans about 90 different AMPs are reported to be present. AMPs in general have two major physical features and they are—a) cationic charge and b) a significant proportion of hydrophobic residues. The cationic charge of the AMPs promotes selectivity for negatively charged microbial surfaces whereas the hydrophobicity facilitates interactions with the cell membrane of the microbial species.

The present inventors have been working to provide hygiene benefits to consumers through the route of enhancing the AMP levels in the skin. They wish to provide this through use of natural molecules which are perceived by the consumers to be more skin friendly and thereby less harsh. Further, they also wish to provide antimicrobial benefits to the body by way of killing or inactivating invading infectious microorganisms.

Specifically, one of the most common and leading causative agents for skin infection is the Staphylococcus aureus, causing various skin and soft tissue infections. Colonization of S. aureus on the host skin surface is the initial stage of infection, on breaching into the deeper tissues, the microbe establishes infection by employing its complex strategies. In particular, S. aureus overcomes the host innate system by producing a range of toxins such as haemolysins, leukotoxins that impairs the cellular membrane and causes lysis.

To achieve the goal of protecting the skin against such microorganisms, various actives were tested and after extensive research, the present inventors found that a commonly used fatty acid viz. hydroxy stearic acid enhances AMP levels on the skin. Further the AMPs so generated interact synergistically with the externally applied hydroxystearic acid to provide antimicrobial activity against infective organisms like S. aureus, P. aeruginosa among others. The antimicrobial benefit was also found to be achieved through a composition comprising a combination of hydroxystearic acid and AMPS and/or AMLs.

Hydroxystearic acid esp. 12-Hydroxystearic acid (12-HSA) has been used in personal care as a part of a fatty acid mixture in cleansing compositions, or as an emulsifier in cosmetics. 12-HSA has extensive use in industrial applications as a lubricant and for production of greases. It is extensively used as a lubricant in the oil drilling industry. To the knowledge of the present inventors its use as an active for generation of AMPs on topical surfaces of the body is not known.

The present inventors have found that on application of hydroxystearic acid on skin (which could be skin per se or scalp or oral cavity), the active induces generation of AMPs which is known to be an important step in improving the immunity of the skin against attack by microorganisms. Thus, the benefit obtained by a consumer is that the skin is protected against germs which may attack in the future by application of a composition comprising hydroxystearic acid.

### SUMMARY OF THE INVENTION

The first aspect of the present invention provides for use of hydroxystearic acid for inducing secretion of anti-microbial peptides (AMPs) when applied on an external surface of the human body.

Another aspect of the present invention provides for a method of providing antimicrobial benefit to an external surface of the body comprising the step of applying a composition comprising hydroxystearic acid and antimicrobial peptides or antimicrobial lipids or mixtures thereof to said external surface.

Yet another aspect of the present invention provides for an antimicrobial composition comprising (i) hydroxystearic acid and (ii) antimicrobial peptides or antimicrobial lipids or mixtures thereof.

### DETAILED DESCRIPTION OF THE INVENTION

These and other aspects, features and advantages will become apparent to those of ordinary skill in the art from a reading of the following detailed description and the appended claims. For the avoidance of doubt, any feature of one aspect of the present invention may be utilized in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated and may be abbreviated as “wt %”. Except in the operating and comparative examples, or where otherwise explicitly indicated, all numbers in this description indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated.

“Skin” as used herein, is meant to include the external surface of mammals, especially humans and includes skin, scalp, hair and oral cavity. The use as per this invention could be by way of applying hydroxystearic acid or the composition as per the invention in a leave-on or in a rinse off product, and includes any product applied to a human body primarily for hygiene benefits but may also improve appearance, cleansing, odor control or general aesthetics. The composition can be in the form of a liquid, lotion, cream, foam, scrub, gel, soap bar or toner, or applied with an implement or via a face mask, pad, wipe or patch. Non-limiting examples of such compositions include leave-on products like skin lotions and creams, antiperspirants, deodorants, depilatories, lipsticks, foundations, mascara, sunless tanners, sanitizers, gels, sprays or sunscreen lotions or through rinse off products like shampoos, conditioners, shower gels, toilet bars, face wash, hand wash or body wash products.

Hydroxystearic acid for use in the present invention preferably induces secretion of AMPs from keratinocytes. The AMPs thus secreted provide for improving the immunity of the external surface of the body. The external surface includes skin, scalp or oral cavity.

It is preferred that the hydroxystearic acid is 10-hydroxystearic acid, 12-hydroxystearic acid or trihydoxystearic acid (e.g. 9,10, 13-trihydroxystearic acid) or trihydroxy stearin or compounds that yield one or more molecules of hydroxystearic acid or hydroxystearate on their breakdown like mono, di or tri ester of glycerol with hydroxystearic acid. Of these, 10-hydroxystearic acid, 12-hydroxystearic acid and 9,10, 13-trihydroxystearic acid are more preferred. 12-hydroxystearic acid (12-HSA) is most preferred. 12-HSA has the structure as given below:

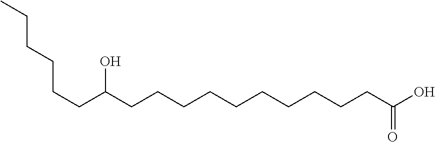 [SMILES: CCCCCCC(O)CCCCCCCCCCC(=O)O]

It has been found by way of the present invention that hydroxystearic acid activates keratinocytes, which are the major cells in the skin epidermis to provide the benefits of the present invention viz. inducing secretion of anti-microbial peptides (AMPs). This causes hydroxystearic acid to boost protection shield against germs i.e improves the immunity of the external surface. Hydroxystearic acid therefore provides protection for the body against infections by boosting the body's own defense. In other words, the active primes the body surface for germ protection. The advantage of this is that it provides long-lasting protection e.g. up to 24 hours of protection against germs.

The use of hydroxystearic acid for inducing secretion of AMPs as per this invention is preferably for cosmetic use i.e. it is for non-therapeutic application.

Yet another aspect of the present invention relates to hydroxystearic acid for prophylactically inducing secretion of anti-microbial peptides (AMPs) when applied on an external surface of the human body.

Yet another aspect of the invention relates to a method of providing protection to an external surface of the body by way of inducing secretion of anti-microbial peptides (AMPs) comprising the step of applying hydroxystearic acid to said external surface. Preferably, the method is non-therapeutic.

According to yet another aspect of the present invention there is provided use of a composition comprising hydroxystearic acid and antimicrobial peptides or antimicrobial lipids or mixtures thereof for antimicrobial benefit. Preferably, the use is non-therapeutic.

According yet another aspect of the present invention there is provided a method of providing antimicrobial benefit to an external surface of the body comprising the step of applying a composition comprising hydroxystearic acid and antimicrobial peptides or antimicrobial lipids or mixtures thereof to said external surface. Preferably, the method is non-therapeutic.

The composition in which hydroxystearic acid is used as per the present invention may comprise the following preferred features. Hydroxystearic acid is preferably present in 0.000001 to 10%, more preferably 0.00001 to 8%, even more preferably 0.0001 to 7%, further more preferably 0.0005 to 5%, still more preferably 0.001 to 4%, yet more preferably 0.01 to 3% and even further more preferably form 0.1 to 1% by weight of the composition.

The composition as per the invention comprises antimicrobial peptides (AMPs) or antimicrobial lipids (AMLs) or mixtures thereof in addition to hydroxystearic acid for getting the desired antimicrobial benefit. Preferably the composition comprises AMLs in addition to hydroxystearic acid. Preferably, the composition is non-therapeutic.

Preferably, AMPs are selected from defensins e.g. human beta defensin 1, human beta defensin 2, human beta defensin 3 and human beta defensin 4; histatins e.g. histatin 5, histatin 7; cathelicidin e.g. LL-37; dermcidin e.g. dermcidin 1; S100 peptides e.g. psoriasin; and RNAse peptides e.g. RNAse7, which may be used in the present invention individually or as a combination. More preferably, AMPs are selected from human beta defensin 1, human beta defensin 2, human beta defensin 3 and human beta defensin 4, LL-37 and mixtures thereof. Even more preferably, the AMP selected is human beta defensin 3, LL-37 and mixtures thereof.

Recent studies have shown a variety of antimicrobial peptides (AMPs) mimic chemokines and have the capacity to rapidly activate host innate and adaptive immune systems by serving as early warning signals. They are capable of recruiting and activating antigen-presenting cells. These potent immunostimulants include defensins, cathelicidin (LL-37), eosinophil -derived neurotoxin (EDN), and high-mobility group box protein 1 (HMGB1). For example, defensins, LL-37, HMGB1 and EDN mimic chemokine and cytokine activities by interacting with CCR6, FPRL-1 and Toll-like receptors (TLR2) respectively. These antimicrobial peptides are constitutively produced and released by leukocytes, keratinocytes and epithelial cells lining the gastrointestinal, genitourinary and tracheobronchial tree. In addition, they are induced by injurious stimulants and cytokines. These peptides all have in vivo immunoadjuvant effects.

These AMPs have now been grouped under the novel term “alarmins”, in recognition of their role in rapidly mobilizing the immune system for protection against host damage.

The antimicrobial lipids (AMLs) which in combination with hydroxystearic acid provide the synergistic antimicrobial activity preferably include sapienic acid, sphingosine, dihydrosphingosine, palmitoleic acid, lauric acid, phytosphingosine or combinations of one or more of the AMLs. More preferably, AML is selected from sphingosine, phytosphingosine, lauric acid and mixtures thereof.

The structures of the various antimicrobial lipids for use in the present invention are given below:

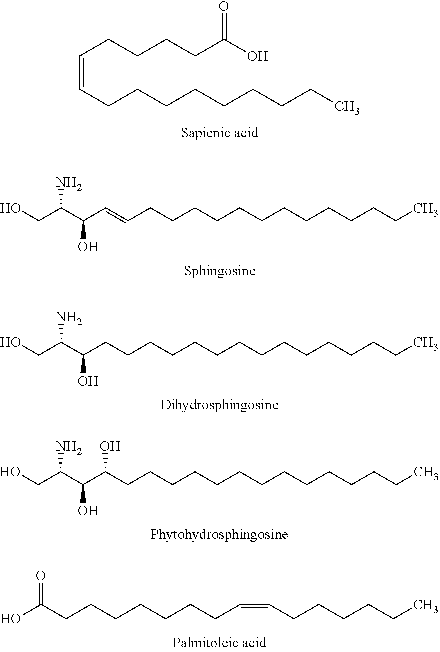 [SMILES: CCCCCCCCC/C=C\CCCCC(=O)O ; CCCCCCCCCCCCC/C=C/[C@@H](O)[C@@H](N)CO ; CCCCCCCCCCCCCCC[C@@H](O)[C@@H](N)CO ; CCCCCCCCCCCCCC[C@@H](O)[C@@H](O)[C@@H](N)CO ; CCCCCC/C=C\CCCCCCCC(=O)O]

Sapienic acid and palmitoleic acid are generally added into the antimicrobial agents in purified form.

Preferably, the composition comprises AMLs or AMPs or mixtures thereof in an amount ranging from 0.000001 to 2%, more preferably from 0.00005 to 1% even more preferably from 0.00001 to 0.1%, further more preferably 0.0001 to 0.05% and still more preferably from 0.001 to 0.01% by weight of the composition.

Preferred combinations of AMLs with hydroxystearic acid include:
12-HSA+lauric acid,
12-HSA+phytosphingosine; and
12-HSA+sphingosine.

Preferred combinations of AMPs with hydroxystearic acid include:
12-HSA+LL-37,
12-HSA+human beta defensin 3.

Preferably, such combinations are selected from:
12-HSA+LL37+Lauric acid,
12-HSA+LL37+Phytosphingosine,
12-HSA+LL37+Sphingosine,
12=HSA+Human beta defensin 3+Lauric acid,
12-HSA+Human beta defensin 3+Phytosphingosine,
12-HSA+Human beta defensin 3+Sphingosine.

The composition preferably comprises a cosmetically acceptable base. The cosmetically acceptable base is preferably a cream, lotion, gel or emulsion. It is especially preferred that the cosmetically acceptable base comprises a surfactant.

The composition of the invention may be prepared so that it is suitable for use as a product for skin cleansing or as an oral care, a skin care, scalp or a hair care product. The product may be delivered in the form of a solid, soft solid, liquid, emulsion, microemulsion, lotion, cream, gel, or aerosol forms.

### Skin Cleansing

By ‘a cleansing composition’ as used herein, is meant to include a composition for topical application to skin, hair and/or scalp of mammals, especially humans for cleansing benefit and such compositions generally include a surfactant. Such a composition is generally applied on to the desired topical surface of the body for a period of time from a few seconds to up to a few minutes generally after diluting with water. After this period of time of application the composition is generally rinsed off with water or wiped away.

The cleansing composition could be in any format either in solid form or in liquid form. When in solid form it is preferably a soap bar.

### Soap Bar:

The soap for preparing the cleansing composition of the invention is preferably a C₈-C₂₄ soap, more preferably C₁₀-C₂₀ soap and most preferably C₁₂-C₁₈ soap. The cation of the soap can be alkali metal, alkaline earth metal or ammonium. Preferably, the cation of the soap is selected from sodium, potassium or ammonium. More preferably the cation of the soap is sodium or potassium. Fatty acids derived from other suitable oils/fats such as groundnut, soybean, tallow, palm, palm kernel, etc. may also be used in other desired proportions.

A cosmetically acceptable base comprising the anionic surfactant forms the rest of the composition other than the water-in-oil emulsion present in the cleansing composition. Thus, the cosmetically acceptable base generally forms 80 to 99% by weight of the cleansing composition.

When present, the anionic surfactant e.g. soap, is preferably present in an amount of 1 to 90%, preferably from 10 to 85%, more preferably 25 to 75% by weight of the cleansing composition. The cleansing composition is preferably in the form of a solid or semi solid form, most preferably in a solid form. Preferred solid compositions are in the shape of a soap bar.

Other anionic surfactants are preferably selected from alkyl ether sulphate, primary alkyl sulphate, secondary alkyl sulphonates, alkyl benzene sulphonates, or ethoxylated alkyl sulphates. The anionic surfactant other than soap which is preferred in the cleansing composition is an alkyl ether sulphate preferably those having between 1 and 3 ethylene oxide groups, either from natural or synthetic source and/or sulphonic acid. Especially preferred are sodium lauryl ether sulphates. Alkyl polyglucoside may also be present in the composition, preferably those having a carbon chain length between C₆ and C₁₆.

Preferred cleansing compositions may include other known ingredients such as perfumes, pigments, preservatives, emollients, sunscreens, gelling agents and thickening agents. Choice of these ingredients will largely depend on the format of the composition. Water is a preferred carrier. When water is present, it is preferably present in at least 1%, more preferably at least 2%, further more preferably at least 5% by weight of the composition. When water is the carrier, a preferred cleansing composition comprises 10 to 50%, more preferably 12 to 40% by weight water.

The composition of the invention may also be delivered through a moisturizing bar or liquid composition. Moisturizing bar compositions comprising fatty acyl isethionates (e.g. cocyl isethionate) are especially preferred.

Fatty acyl isethionates (e.g., cocoyl isethionates) surfactant “products” are defined as mixtures of anionic acyl isethionate surfactants and fatty acids/fatty acid soaps. They are highly desirable in personal care skin or hair cleansing products, particularly in personal care products, because they lather well, are mild to the skin and have good emollient properties. Typically, fatty acid isethionate surfactant products are produced by esterification of fatty acids or by reaction of fatty acid chloride having carbon chain length of C8 to C20 with isethionate. A typical surfactant product containing fatty acyl isethionate contains about 40 to 95 wt. % acid isethionate, and 5 to 50 wt. %, typically 10 to 40 wt. % free fatty acid, in addition to isethionate salts, typically at less than 5%, and trace (less than 2 wt. %) of other additives. Fatty acid soap may be included in the range of 5 to 15 wt %. Other surfactants like betaines may be included in 1 to 5 wt %. Water is generally included in 2 to 8 wt % of the composition.

### Liquid Cleansing Compositions:

The liquid cleansing composition of the invention is generally to be used for personal cleansing. It is preferably a composition for cleaning of topical surfaces body, hand or face which comprises surfactants at low concentration and are mild on skin. The surfactant is generally included in 1 to 30%, preferably 4 to 18%, more preferably 6 to 12% by weight of the liquid cleansing composition.

Surfactants for inclusion in the liquid cleansing composition of the invention may preferably be of the anionic, non-ionic, cationic or amphoteric types. A useful surfactant for inclusion in the liquid cleansing composition of the invention is sodium lauryl ether sulphate (SLES). The SLES for use in the present invention generally preferably has 1 to 3 Ethoxylate (EO) groups. SLES is preferably included in 3 to 15% by weight of the composition. The other surfactant which may be included in the present invention is cocoamide monoethanol amine (CMEA). CMEA is preferably included in 1 to 5% by weight of the composition. One preferred aspect of the liquid cleansing composition of the invention relates to an aspect wherein the surfactant comprises a mixture of sodium lauryl ether sulphate (SLES) and coco amide monoethanol amine (CMEA).

Another useful surfactant for inclusion in the liquid cleansing composition of the invention is an amphoteric surfactant preferably a betaine surfactant, more preferably an alkyl amidopropyl betaine surfactant for example cocamidopropyl betaine. In a preferred embodiment, the composition comprises from 0.1 to 5 wt. %, preferably from 0.5 to 4 wt. %, more preferably from 1 to 3 wt. % of a betaine surfactant.

Surfactants of the non-ionic class for inclusion in the liquid cleansing composition of the invention are preferably of the polyoxyethylene sorbitan alkyl esters class (sold as Tween surfactants)), fatty alcohol ethoxylates (sold as Brij surfactants), alkyl phenol ethoxylates (sold as Triton surfactants), fatty acid ethoxylates (sold as Myrj surfactants), and Alkyl poly glucosides (sold as Plantacare surfactants).

Water is a preferred carrier in liquid cleansing compositions of the invention. In such compositions, water is generally present in 70 to 95% by weight. Preferred liquid cleansing compositions may include other known ingredients such as electrolytes, perfumes, pigments, preservatives, emollients, sunscreens, emulsifiers, gelling agents and thickening agents.

The invention may also be used in intimate hygiene products esp. for use by women. Such products are generally available as a liquid with certain amount of surfactants and emollients formulated at a pH which is mild on women when used for obtaining intimate hygiene. Such products are also known as feminine hygiene products or menstrual hygiene products. Such liquid products generally comprise high amount of water in the range of 40 to 80 wt %, preferably 50 to 70 wt %. They further comprise surfactants e.g. an anionic and/or amphoteric surfactants. Such surfactants are preferred to be included in 10 to 50 wt % preferably 20 to 40 wt % of the composition. As especially preferred surfactant for use in such products is triethanolamine lauryl sulphate, ammonium lauryl sulphate, cocoamidopropyl betaine or mixtures thereof. Additionally, the product may comprise oils, emollients, thickeners and other additives to aid in giving a mild sensation to the skin. Carboxylic acids like lactic acid is generally included in such compositions to provide additional hygiene benefits. The pH of such products is generally in the range of 2.5 to 6.0 preferably in the range of 2.5 to 4.5.

### Oral Care

When the personal care composition is delivered for oral care, it includes a cosmetically acceptable base which may be an abrasive, a thickener, a humectant or an orally acceptable surfactant. The product may be delivered in the form of an ointment, a gel, a dentifrice or a mouthwash. Oral care compositions preferably comprise an abrasive. Gels usually contain silica, whereas opaque creams generally contain calcium based abrasives, especially chalk.

Preferred toothpaste compositions have 5 to 60 wt % calcium based abrasive.

In a preferred embodiment, the composition comprises a thickener. Typically, thickening silica, sodium carboxymethyl cellulose and/or a Carbomer is/are preferred thickeners for use in the composition of the invention. Thickener, when present, preferably makes up from 0.01 to about 10%, more preferably from 0.1 to 9%, and most preferably, from 1.5 to 8% by weight of the composition.

Suitable humectants are preferably used in the oral care composition of the present invention. Glycerin, polyethylene glycol, sorbitol or mixtures thereof are the preferred humectants.

The humectant may be present in the range of from 10 to 90% by weight of oral care compositions. Preferably, an oral care composition comprises a surfactant. Preferably the composition comprises at least 0.01% surfactant by weight of the composition, more preferably at least 0.1% and most preferably from 0.5 to 7%. The preferred anionic surfactants are sodium lauryl sulphate and/or sodium dodecylbenzene sulfonate. Most preferably the surfactant is sodium lauryl sulphate.

Water may preferably be included in 5 to 95%, in particular 10 to 75%, and especially at from 10 to 60%, further more preferably 10 to 45% by total weight of the composition.

The oral care composition of the present invention may contain a variety of other ingredients which are common in the art to enhance physical properties and performance. These ingredients include antimicrobial, anti-caries agents, plaque buffers, fluoride sources, vitamins, plant extracts, desensitizing agents, anti-calculus agents, biomolecules, flavors, proteinaceous materials, preservatives, opacifying agents, coloring agents, pH-adjusting agents, sweetening agents, particulate abrasive materials, polymeric compounds, buffers and salts to buffer the pH and ionic strength of the compositions, and mixtures thereof. Such ingredients typically and collectively make-up less than 20% by weight of the composition, and preferably, from 0.0 to 15% by weight, and most preferably, from 0.01 to 12% by weight of the composition, including all ranges subsumed therein.

### Hair Care

As per a preferred aspect of the invention, the composition may be used for hair care or cleansing. One medium through which this may be delivered is that of a shampoo. The composition of the invention especially shampoos are formulated with an anionic surfactant e.g. an alkyl sulphate and/or ethoxylated alkyl sulfate surfactant. These anionic surfactants are preferably present at a level of from 1 to 20%. Preferred alkyl sulfates are C8-18 alky sulfates, more preferably C12-18 alkyl sulfates, preferably in the form of a salt with a solubilising cation such as sodium, potassium, ammonium or substituted ammonium. An example is sodium lauryl ether sulfate (SLES). Preferred ethoxylated alkyl sulfate anionic surfactant is sodium lauryl ether sulfate (SLES). SLES having an average degree of ethoxylation of from 0.5 to 3, preferably 1 to 3 is especially preferred. Shampoos preferably additionally comprise an amphoteric surfactant preferably a betaine surfactant preferably an alkyl amidopropyl betaine surfactant for example cocamidopropyl betaine. In a preferred embodiment, the composition comprises from 0.1 to 10 wt. %, preferably from 0.5 to 8 wt. %, more preferably from 1 to 5 wt. % of a betaine surfactant.

To enhance deposition of actives from compositions of the invention especially shampoos, cationic polymers are generally included therein. In the present invention too, it is preferred that the composition additionally includes 0.01 to 2.0% of a cationic polymer. The cationic polymer is preferably guar hydroxypropyl trimonium chloride. When conditioning benefits are to be delivered through the composition of the invention the composition is called a hair conditioner. Typically, the most popular conditioning agents used in hair care compositions are water-insoluble oily materials such as mineral oils, naturally occurring oils such as triglycerides and silicone polymers.

Conditioning benefit is achieved by the oily material being deposited onto the hair resulting in the formation of a film, which makes the hair easier to comb when wet and more manageable when dry. An especially useful conditioning agent is a silicone compound, preferably a non-volatile silicone compound. The pH of the composition is preferably equal to or higher than 4.0, more preferably in the range of 5.0 to 7.0.

The hair conditioning composition usually comprises conditioning surfactants selected from cationic surfactants, used singly or in admixture. Particularly useful cationic surfactant for use in conditioners according to the invention is cetyltrimethylammonium chloride, behenyltrimethylammonium chloride or stearamidopropyl dimethylamine. Hair conditioning compositions of the invention preferably may also additionally comprise a fatty alcohol.

### Skin Care

The composition of the invention may be used for skin care. By skin care is meant that the composition is applied on the skin and left thereon till the person goes for a shower or a bath usually after several hours or after about a day. The cosmetically acceptable base is generally present in an amount ranging from 10 to 99.9%, more preferably from 40 to 85% by total weight of the composition. It is particularly preferred that the cosmetically acceptable base includes water. Water is preferably included in an amount from 30 to 90%, more preferably from 30 to 85%, most preferably from 30 to 80% by total weight of the composition. Besides water, suitable carrier classes include silicones, polyhydric alcohols, hydrocarbons, triglycerides and thickening powders.

The skin care composition of the invention may be in any form including toners, lotions, creams, mousses, scrub, serum or gel that is suitable for topical application to the skin. It is preferred that the composition is a skin lotion or a cream.

The composition may comprise an emollient oil that act as a co-solvent. Suitable emollient oils include, for example, ester of alkoxylated aromatic alcohol with fatty carboxylic acid, esters of polyglycols or diols with fatty carboxylic acid such as caprylic/capric triglyceride, ester of fatty alcohol and fatty acid, alkoxylated derivative of benzyl alcohol and mixtures thereof. Preferably the emollient oil is caprylic/capric triglyceride.

Typically, such compositions comprise co-solvent in an amount from 0.01 to 10%, more preferably from 0.1 to 8%, most preferably from 1 to 6%, based on the total weight of the sunscreen composition and including all ranges subsumed therein.

The composition may additionally comprise sunscreen agents such as inorganic sunscreens or an organic sunscreen. Examples of organic sunscreens are zinc oxide, titanium dioxide, iron oxide, silica such as fumed silica. The composition of the invention may comprise a UV-A sunscreen agent selected from the group consisting of a dibenzoylmethane derivative, a triazine derivative, a benzophenone derivative and mixtures thereof. The composition of the invention may also comprise a UV-B sunscreen agent. Suitable UV-B sunscreen agent of the invention is selected from the group consisting of a benzophenone, an anthranilate, a salicylate, a cinnamate, a camphor, benzylidene malonate, a triazone, and derivatives thereof.

A skin lightening agent may also be incorporated into the composition of the invention.

Skin care products also include sanitizers that ensure almost instant kill of microorganisms present on the skin. Sanitizers preferably comprise 20 to 90%, preferably 30 to 80% by wt. % of C₂ to C₄ monohydric alcohols wherein said monohydric alcohols are selected from ethanol, isopropyl alcohol or combinations. The sanitizer composition may comprise a combination of ethanol and isopropyl alcohol.

Most preferably the alcohol is preferably 40 to 75 wt. % and optimally the composition comprises 55 to 70 wt. % of the C₂ to C₄ monohydric alcohols.

Preferably, sanitizer compositions may contain 30 to 80 wt. %, more preferably 40 to 70 wt. % and most preferably 50 to 68 wt. % ethanol. Sanitizer may include certain polyols. Polyols in the composition according to the invention are preferably sorbitol, glycerol, polyethylene glycol, propylene glycol and combinations thereof.

Sanitizers preferably comprise 0.01 to 1 wt. % of a thickening agent. More preferably the composition comprises 0.05 to 0.8 wt. % of a thickening agent, most preferably 0.08 to 0.2 wt. % of a thickening agent. Water is generally present in a sanitizer composition from 10 to 40 wt. %. More preferably the composition comprises 12 to 35 wt. % of water, most preferably 25 to 30 wt. % of water.

### Deodorant Products

A specific class of skin care compositions is what is known as deodourant compositions. These compositions are usually applied to areas of the skin which generate body odour like the underarm region. These products work by either masking the odour or by ensuring that odour causing molecules are not generated or by killing or inactivating the odour causing microorgansims. A class of such products are also known as anti-perspirant products as they reduce or minimize the amount of sweat that is generated by the person.

These products can be applied cosmetically and topically to the skin, broadly speaking, by one of two methods. In one method, sometimes called a contact method, a composition is wiped across the surface of the skin, depositing a fraction of the composition as it passes. In the second method, sometimes called the non-contact method, the composition is sprayed from a dispenser held proximate to the skin. The spray can be developed by mechanical means of generating pressure on the contents of the dispenser, such as a pump or a squeezable sidewall or by internally generated pressure arising from a fraction of a liquefied propellant volatilising, the dispenser commonly being called an aerosol.

There are broadly speaking two classes of contact compositions, one of which is liquid and usually applied using a roll-on dispenser or possibly absorbed into or onto a wipe, and in the second of which the desired active is distributed within a carrier liquid that forms a continuous phase that has been gelled. In one variation, the carrier fluid comprises a solvent for the desired active and in a second variation, the active remains as a particulate solid that is suspended in an oil, usually a blend of oils.

### Stick or Soft Solid Compositions:

Many different materials have been proposed as gellant for a continuous oil phase, including waxes, small molecule gelling agents and polymers, They each have their advantages and of them, one of the most popular class of gellant has comprised waxes, partly at least due to their ready availability and ease of processing, including in particular linear fatty alcohol wax gellants. A gelled deodourant composition is applied topically to skin by wiping it across and in contact with the skin, thereby depositing on the skin a thin film.

### Roll-on:

Liquid compositions that are applicable from a roll-on broadly speaking can be divided into two classes, namely those in which an active is suspended in a hydrophobic carrier, such as a volatile silicone and those in which the active is dissolved in a carrier liquid. The latter has proven to be more popular. There are mainly two sorts of dissolving carrier liquid, namely carriers that are predominantly alcoholic, which is to say the greater part of the dissolving carrier fluid comprises ethanol and the second class in which the carrier liquid is mainly water. The former was very popular because ethanol is a mild bactericide in its own right, but its popularity waned because it stings, especially if the surface onto which the composition has been applied has been damaged or cut, such as can easily arise during shaving or other de-hairing operations.

The second class of formulations that is an alternative to alcoholic formulations comprise a dispersion of water-insoluble or very poorly water soluble ingredients in an aqueous solution of the active. Herein, such compositions will be called emulsions. Roll-on emulsions commonly comprise one or more emulsifiers to maintain a distribution of the water-soluble ingredients.

### Aerosol Compositions:

Deodorant compositions may be delivered through an aerosol which comprises a propellant in addition to the other ingredients described hereinabove.

Propellants herein generally accord with one of three classes; i) low boiling point gases liquifided by compression, ii) volatile ethers and iii) compressed non-oxidising gases. Class i) is conveniently a low boiling point material, typically boiling below −5° C., and often below −15ºC, and in particular, alkanes and/or halogenated hydrocarbons. This class of propellant is usually liquefied at the pressure in the aerosol canister and evaporates to generate the pressure to expel the composition out of the canister. Examples of suitable alkanes include particularly propane, butane or isobutene. The second class of propellant comprises a very volatile ether of which the most widely employed ether hitherto is dimethyl ether. This propellant can advantageously be employed at relatively low weight ratio of propellant to base formulation, for example to as low as 5:95. It can also be employed in admixture with, for example, compressible/liquefiable alkane gasses. The third class of propellant comprises compressed non-oxidising gasses, and in particular carbon dioxide or nitrogen. Inert gases like neon are a theoretical alternative.

Skin care compositions may also comprise other ingredients which are common in the art to enhance physical properties and performance. Suitable ingredients include but are not limited to humectants, thickeners, opacifiers, binders, colorants and pigments, pH adjusting agents, preservatives, optics, perfumes, viscosity modifiers, biological additives, buffering agents, conditioners, essential oils and skin benefit agents including anti-inflammatory agents, cooling agents, antiperspirant agents, anti-aging agents, anti-acne agents, anti-microbial agents and antioxidants.

The invention is now further described by way of the following non-limiting examples.

### EXAMPLES

### Examples 1-5: Effect of 12-HSA (at 1 μM Concentration) on AMP Gene Expression

The following protocol was used for evaluating actives (12-HSA) on keratinocytes for AMP gene expression

### Materials:

Normal Human Epidermal Keratinocyte cells (NHEK), EpiLife Keratinocyte Growth Medium (KGM) (with antibiotics Pen-Strep), Dulbecco's Phosphate Buffered Salone (DPBS), Trypsin-EDTA, Trypsin Neutralizer Solution, Ethanol, RNA extraction kit (RNeasy kit), RLT buffer, RT-PCR kit.

### Procedure:

1. Thaw the NHEK cells and expand them in KGM in 2-25 cc and then 75 cc flasks to 80% confluency, changing media every alternate day and incubating at 37° C. in a 5% CO₂ incubator.
2. Discard spent media and rinse the adhered cells in the flask with DPBS. Add Trypsin-EDTA and tap to dislodge the adhered cells. Add Trypsin Neutralizer and gently mix it.
3. Transfer the contents to a 15-ml tube and spin down the cells. Discard supernatant and resuspend cells in KGM. Count and seed 30,000-50,000 cells per well in 24 well TC plates, incubate to 80-85% confluence (˜2-3 days). Change media every alternate day. Add differentiation media (KGM with 2 mM CaCl₂) and incubate for 2-3 days.
4. Prepare actives in KGM at the required concentration (final concentration of ethanol solvent, if used, should be less than 0.1%).
5. Replace the differentiation media with the actives containing media and incubate for 24 h
6. Discard the supernatant and wash cells once with DPBS. Add 250 μL of RLT buffer to each well to lyse the cells and transfer the entire content to 1.5 ml tubes. Store at −80° C. until extraction.
7. Perform RNA extraction as per the kit, quantify the RNA, do cDNA synthesis and do real time qPCR for cDNA samples. Calculate the relative expression level over control normalised to housekeeping gene.

Using the above protocol the expression of AMP gene was measured and the data is summarised in Table-1 below.

TABLE 1
AMP gene expression with 12HSA at 1 μM
(=0.00003 wt %) concentration

| | | Fold change with |
|---|---|---|
| Example | AMP Gene | respect to control |
| 1 | Control | 1.00 |
| 2 | S100A7 | 1.66 |
| 3 | RNase7 | 2.56 |
| 4 | LL-37 | 2.09 |
| 5 | HBD-3 | 2.16 |

The fold change is an indication of the amount of gene expressed in comparison to the control where no active (12-HSA) was used. Thus, for example, a sample with 12-HSA caused expression of the gene S100A7 1.66 times more than the sample without 12-HSA. The data in the above table therefore indicates that very many AMPs genes are expressed many fold higher than control when 12-HSA is used on keratinocytes.

### Examples 6-10: Anti Microbial Peptide Expression From 12-HSA Treatments at Various Concentrations

Psoriasin (S100A7) AMP from the above list was chosen to study the effect of concentration of 12-HSA on the secretion of the AMP. The procedure used was as follows:

### Protocol for Evaluating Active (12-HSA) on Keratinocytes for AMP Secretion

### Materials:

Normal Human Epidermal Keratinocyte cells (NHEK), EpiLife Keratinocyte Growth Medium (KGM) (with antibiotics Pen-Strep), Dulbecco's Phosphate Buffered Salone (DPBS), Trypsin-EDTA, Trypsin Neutralizer Solution, Ethanol, Psoriasin ELISA Kit.

### Procedure:

1. Thaw the NHEK cells and expand them in KGM in 2 -25 cc and then 75 cc flasks to 80% confluency, changing media every alternate day and incubating at 37° C. in a 5% CO₂ incubator.
2. Discard spent media and rinse the adhered cells in the flask with DPBS. Add Trypsin-EDTA and tap to dislodge the adhered cells. Add Trypsin Neutralizer and gently mix it.
3. Transfer the contents to a 15-ml tube and spin down the cells. Discard supernatant and resuspend cells in KGM. Count and seed 30,000-50,000 cells per well in 24 well TC plates, incubate to 80-85% confluence (˜2-3 days). Change media every alternate day. Add differentiation media (KGM with 2 mM CaCl₂) and incubate for 2-3 days.
4. Prepare actives in KGM at the required concentration (final concentration of ethanol solvent, if used, should be less than 0.1%).
5. Replace the differentiation media with the actives containing media and incubate for 72 h
6. Collect supernatant and store at −80° C. Thaw them completely before performing ELISA to measure AMP (Psoriasin).
7. For ELISA, run the required standards and samples (undiluted and diluted) and using standard plot, the amount of AMP in samples calculated.

Using the above protocol the expression of the peptide was measured and the data is summarised in Table-2 below.

TABLE 2

| | | Fold change with respect |
|---|---|---|
| | | to control(Psoriasin |
| Example | Concentration of 12-HSA | increase) |
| 6 | Control | 1.00 |
| 7 | 0.25 μM | 8.12 |
| 8 | 0.50 μM | 15.07 |
| 9 | 1.0 μM | 9.79 |
| 10 | 2.0 μM | 15.70 |

The data in the above table indicates that 12-HSA causes expression of AMPs from keratinocytes in significant amounts and this is observed over a large range of concentrations.

It is to be understood that the experiments described herein were conducted in an in-vitro assay to evaluate the synergistic antimicrobial effect. It is expected that the concentrations to be actually used to prepare a composition for topical use could be orders of magnitude higher due to the following reasons that affect the difference in concentration in the bulk as compared to that at the cellular level. The composition may be formulated as an emulsion or a gel with very many additional ingredients. The concentration of the desired actives in the oil phase and in the water phase, is expected to be very different. They may also have very different physical and hydrodynamic properties like partition coefficients, diffusional rates, convective transport rates, rheological properties etc. Therefore it is expected that the concentrations to be used when formulated as a composition would be very different from that at the cellular level.

### Examples 11-26: Synergy of 12-HSA with AMLs or AMPs or Mixtures Thereof in Providing Anti-Microbial Efficacy

The antimicrobial efficacy of a combination of 12-HSA at 10 μM (=0.0003 wt %) with a combination of AML (phytosphingosine) at 1 μg/ml (=0.0001 wt %) and AMP (LL-37) at 0.5 μg/ml (=0.00005 wt %) against two organisms S. aureus and P. aeruginosa was studied. The experiments were carried out using the following assay.

### Microdilution Assay

Materials: Tryptic Soy Agar (TSA) medium, AMP stock (LL37), AML stock (Phytosphingosine), Test microbe (S. aureus, P. aeruginosa), Phosphate buffer (pH 5.8), D/E medium.

### Method:

1. Inoculate test culture in a TSA plate and incubate for 18-24 h at 37° C. in an incubator.
2. Scrape the culture and resuspend in 10 mM Phosphate buffer, adjust cell number to 1×10\hat{ }8 cfu/mL.
3. Serially dilute the culture in 10 mM phosphate buffer to obtain 1×10\hat{ }6 cfu/mL.
4. Prepare 10-100 fold concentration stocks of actives (in water, ethanol or DMSO), LL37 (in MilliQ water) and Phytosphingosine (in ethanol).
5. In a 96-well microtiter plate, add buffer, active LL37 & Phytosphingosine, and make up the total volume to 165 μL with water. Keep appropriate controls.
6. Add 135 μL of diluted culture, mix and incubate at 37° C. for 4 h.
7. After incubation, remove aliquots from each of the reactions and neutralise by diluting them 1:10 in D/E medium.
8. Dilute further in D/E medium and plate dilutions on TSA plates. Incubate at 37° C. for 24 h.
9. Next day count the colonies and calculate the recovery and log reduction as compared to control.

The data on log number (recovery) of the various samples is given in the Table-3 and 4 below.

The hydroxystearic acid, AMPs and AMLs used in the examples below were sourced commercially from the suppliers and catalogue numbers as indicated below. 12-HSA (TCI; H0308), LL-37 (Tocris; 5213/1), phytosphingosine (TCI; P1765), sapienic acid (Chemscene; CS-0105451), Sphingosine (Sigma; S9666), palmitoleic acid (Sigma; P9417). The bacteria mentioned in the tables below along with their American Type Culture Collection (ATCC) numbers were sourced from Chromachemie.

TABLE 3
Antimicrobial kill against S. aureus ATCC 6538

| | | Recovery (log |
|---|---|---|
| Example | Sample | number) |
| 11 | Control | 5.8 |
| 12 | 10 μM 12-HSA | 4.9 |
| 13 | Phytosphingosine (1 μg/ml) + | 4.5 |
| | LL-37 (0.5 μg/ml) | |
| 14 | 10 μM 12-HSA + | 3.9 |
| | Phytosphingosine (1 μg/ml) + | |
| | LL-37 (0.5 μg/ml) | |

TABLE 4
Antimicrobial kill against P. aeruginosa ATCC15442

| | | Recovery (log |
|---|---|---|
| Example | Sample | number) |
| 15 | Control | 6.3 |
| 16 | 10 μM 12-HSA | 6.1 |
| 17 | Phytosphingosine (1 μg/ml) + | 5.9 |
| | LL-37 (0.5 μg/ml) | |
| 18 | 10 μM 12-HSA + | 5.5 |
| | Phytosphingosine (1 μg/ml) + | |
| | LL-37 (0.5 μg/ml) | |

The data in Tables-3 and 4 indicates that the combination of 12-HSA along with an AML and AMP gives synergistic antimicrobial activity as compared to that achieved with each active individually.

TABLE 5
Antimicrobial kill against S. aureus ATCC 6538

| | | Recovery (log |
|---|---|---|
| Example | Sample | number) |
| 19 | Control | 6.59 |
| 20 | 0.005% 12-HSA | 6.26 |
| 21 | 1.0 μg/ml LL-37 | 5.48 |
| 22 | 0.005% 12-HSA + 1.0 μg/ml | 4.62 |
| | LL-37 | |

TABLE 6
Antimicrobial kill against P. aeruginosa ATCC 15442

| | | Recovery (log |
|---|---|---|
| Example | Sample | number) |
| 23 | Control | 6.3 |
| 24 | 0.0003% 12-HSA | 6.1 |
| 25 | 1.0 μg/ml phytosphingosine | 5.9 |
| 26 | 0.0003% 12-HSA + 1.0 μg/ml | 5.4 |
| | Phytosphingosine | |

1.-6. (canceled)

7. A method of providing protection to an external surface of the body by way of inducing secretion of anti-microbial peptides (AMPs) comprising the step of applying hydroxystearic acid to said external surface.

8. (canceled)

9. A method of providing antimicrobial benefit to an external surface of the body comprising the step of applying a composition comprising hydroxystearic acid and antimicrobial peptides or antimicrobial lipids or mixtures thereof to said external surface.

10. An antimicrobial composition comprising (i) 0.000001 to 10% by weight hydroxystearic acid and (ii) 0.000001 to 2% by weight antimicrobial peptide or antimicrobial lipid or mixtures thereof.

11. The antimicrobial composition as claimed in claim 10, wherein the composition comprises (i) 0.000001 to 10% by weight hydroxystearic acid and (ii) 0.000001 to 1% by weight antimicrobial lipid.

12. The antimicrobial composition as claimed in claim 10, comprising a cosmetically acceptable base.

13. The antimicrobial composition as claimed in claim 12, wherein the cosmetically acceptable base is a cream, lotion, gel or emulsion.

14. The antimicrobial composition as claimed in claim 12, wherein the cosmetically acceptable base comprises a surfactant.

15. The method as claimed in claim 7, wherein the hydroxystearic acid is 12-hydroxystearic acid.

16. The method as claimed in claim 7, wherein the external surface includes skin, scalp or oral cavity.

17. The method as claimed in claim 7, wherein the hydroxystearic acid induces secretion of AMPs from keratinocytes.

18. The method as claimed in claim 7, wherein immunity of the external surface is improved.
